(19) United States
(12) Patent Application Publication
(10) Pub. No.: US 2009/0023807 A1
(43) Pub. Date: Jan. 22, 2009

(54) Treatment of Cytokine Dysregulation by Using Sn-2 Gamma-Linolenoyl, Gamma-Diho-Molinolenoyl or Arachidonoyl Patty Acid Glycerol Monoesters

(75) Inventors: Laurence S. Harbige, Kent (GB); Michael J. Leach, Kent (GB); Paul Barraclough, Kent (GB)

Correspondence Address: NIXON & VANDERHYE, PC, 901 NORTH GLEBE ROAD, 11TH FLOOR, ARLINGTON, VA, 22203, US

(73) Assignee: BTG International Limited

(21) Appl. No.: 11/885,255

(22) PCT Filed: Mar. 2, 2006
(86) PCT No.: PCT/GB06/00778

§ 371 Date: Apr. 16, 2008

(30) Foreign Application Priority Data
Mar. 2, 2005 (GB) 0504333.6

(51) Int. Cl. A61K 31/23; A61K 31/231; A61P 37/00; A61P 25/00; A61P 25/28

(52) U.S. Cl. 514/549; 514/560

(57) ABSTRACT

A method of treating a patient in need of therapy for a cytokine dysregulation comprising administering to that patient a therapeutically effective dose of a monoglyceride or metabolic precursor thereof of general formula (I), wherein R¹ is the fatty acyl group of an essential polyunsaturated fatty acid selected from γ-linolenoyl, γ-dihomolinolenoyl and arachidonoyl.

The present invention relates to a method for treating diseases and disorders in which cytokines are in state of imbalance or otherwise capable of modulation to provide therapeutic benefit. Particularly the invention provides a method of treatment of patients in need of therapy for disorders where the cytokines TGF-β1, IL1-β, IL4, IL5, IL6, IL8, IL10, IL13, and γ-IFN are dysregulated or capable of modulation to provide therapeutic benefit. The invention also provides modulation of TNF-α by known compounds previously unused for this purpose.

Particular diseases that are treatable by the present method are disorders such as abnormalities of the immune system, for example systemic lupus erythematosus (SLE), allergy, asthma, crohn's disease and rheumatoid arthritis, but particularly multiple sclerosis, and also neurodegenerative diseases such as sequelae of stroke, head trauma, bleeds and the chronic abnormalities of Alzheimer's and Parkinson's disease. Further disorders that can be pretreated both prophylcatically and therapeutically are coronary heart disease (CHD) abnormalities of pre-mature infants and sepsis.

The inventor's copending patent application PCT/GB2004/002089 and unpublished PCT/GB2004/003524, incorporated herein by reference, relate to the use of synthetic, plant and fungal oils for the treatment of neurodegenerative diseases, particularly multiple sclerosis, stroke, head trauma, Alzheimer's and Parkinson's disease. PCT/GB2004/002089 relates to oils characterised by having at high percentages of the essential fatty acid γ-linolenic acid (GLA) at the sn-2 position of their lipids, typically being over 40% of the sn-2 fatty acid total of the oil. PCT/GB2004/003524 relates to structured lipids having an sn-2 fatty acid residue selected from γ-linolenic acid (GLA), dihomo-γ-linolenic acid (DHGLA) and arachidonic acid (AA).

It is well reported in the literature that essential fatty acids (EFAs) of the n-3 and n-6 unsaturation pattern have beneficial effect in a wide variety of human physiological disorders, including autoimmune disease (WO 02/02105). Harbige (1998) Proc. Nut. Soc. 57, 555-562 reviewed the supplementation of diet with n-3 and n-6 acids in autoimmune disease states, and particularly noted evidence of benefit of γ-linolenic (GLA) and/or linoleic acid (LA) rich oils.

Cytokines are implicated in the pathogenesis of MS, with many studies showing an increase in myelinotoxic inflammatory cytokines (TNF-α, IL-1β and IFN-γ) coinciding with the relapse phase of the disease. Conversely, levels of the anti-inflammatory and immunosuppressive cytokine transforming growth factor-beta1 (TGF-β1) appear to be reduced during a phase of relapse and increase as the patient enters remission. Thus the balance between biologically active TGF-β1 and the pro-inflammatory TNF-α, IL-1β and IFN-γ appears to be dysregulated during MS relapse-remission.

During natural recovery phase from EAE, TGF-β1-secreting T-cells inhibit EAE effector cells, TGF-β1 is expressed in the CNS and, in oral-tolerance-induced protection in EAE, TGF-β and PGE₂ are expressed in the brain (Karpus & Swanborg (1991); Khoury et al (1992)). Harbige ((1998) concluded that dietary γ-linolenic acid effects on EAE are mediated through Th₃-like mechanisms involving TGF-β1 and possibly through superoxide dismutase antioxidant activity.

Borage oil (typically 20% to 23% γ-linolenic acid and 34 to 40% linoleic acid per 100% fatty acid content) and Mucor javanicus fungal oil (see FIG. 1) have been shown to be effective in the EAE animal model used to identify MS candidates, whilst never having been shown to be significantly effective in the human disease. High levels of linoleic rich oil containing low levels of γ-linolenic acid (EPO:linoleic acid:γ-linolenic acid 7:1) partially suppressed the incidence and severity of EAE in rat (Mertin & Stackpoole, 1978) whereas the Bates' Naudicelle study referred to above led to worsening of patients. In spite of the use of Borage oil and other GLA/LA containing oils such as Evening Primrose oil by multiple sclerosis sufferers over the past 30 years or so, the vast majority of patients fail to recover from the disease, showing no significant improvement, with the underlying disease continuing to progress to death.

It has been suggested to use, inter alia, γ-linolenic acid and linoleic acid rich Borage oil as a means to provide immuno-suppression in multiple sclerosis (U.S. Pat. No. 4,058,594). Critically, the dose suggested is 2.4 grams of oil per day and no actual evidence of efficacy is provided. This is much lower than the low 5 g/day dose found to be ineffective in vivo in man in the PCT/GB04/002089 study.

Other more dramatic immunosuppressant treatments, including T cell depleters and modulators such as cyclophosphamide, are also shown to be effective in the EAE model, but where these are employed in the human multiple sclerosis disease symptoms improve, but the underlying disease continues to progress. T-cells indeed produce beneficial cytokines, such as TGF-β1, as well as deleterious ones in man. David Baker of Institute of Neurology, UK summed up the disparity between what is effective in the EAE and in MS with a paper entitled ‘Everything stops EAE, nothing stops MS’ at the 10 May 2004 UK MS Frontiers meeting of the UK MS Society.

In the PCT/GB04/002089 study the present inventors have surprisingly determined that with compliance to a ‘high dose’ treatment with triglyceride oil containing high levels of sn-2 γ-linolenic acid (>40% of residues at the sn-2 being of γ-linolenic acid) with suitable accompanying fatty acid content, remarkable levels of improvement in almost all symptoms of MS can be achieved, way surpassing that provided by the current gold standard treatment. Such success is particularly surprising in the light of the prior use of other γ-linolenic acid containing preparations without success, such as the Naudicelle study.

The PCT/GB04/002089 study shows that over an 18-month period, patients taking high dose (15 g/day) selected high sn-2 γ-linolenic acid borage oil showed significant (p<0.001) and marked improvements in EDSS score, a reduced rate of relapse, symptomatic relief of muscle spasticity and painful sensory symptoms, and improved objective measures of cognitive functions. Low doses of 5 g/day of this borage oil were without effect.

Patients taking the highest dose of this borage oil maintained their level of peripheral blood mononuclear cell production (PBMC) of TGF-β1 during the trial period, their pro-inflammatory cytokines TNF-α and IL-1β were significantly and markedly (<70%) reduced and they either maintained or increased the PBMC membrane long chain omega-6 fatty acids dihomo-γ-linolenic acid (DHLA) and arachidonic acid (AA) in contrast to patients taking placebo who demonstrated loss of these fatty acids over the course of the trial period.

Thus whilst immuno-suppression would be expected to reduce increase of active lesioning and neurodegeneration, the high sn-2 GLA oil treatment apparently targeted maintenance and/or increase of key membrane lipid components that are otherwise specifically lost in MS, being consistent with a correction of a metabolic defect not otherwise effectively treated by current therapies. The fact that the low dose (5 grams/day) had no effect on this supports such determination.

γ-Linolenic acid (18:3 n-6, or GLA) is known to be rapidly converted to longer-chain omega-6 polyunsaturated fatty acids dihomo-γ-linolenic acid and arachidonic acid in vivo (Phylactos et al 1994, Harbige et al 1995, 2000). Therefore to determine how to increase the level of membrane long chain omega-6 fatty acids in MS the inventors have reviewed their results obtained with several GLA-containing oils:—both fungal (from Mucor javanicus) and plant (Borago officianalis), Evening primrose Oenothera spp. or Blackcurrant Ribes spp) as well as a synthetic tri-GLA oil as GLA delivery systems in an in vivo experimental animal model of MS known as chronic relapsing experimental autoimmune encephalomyelitis (CREAE).

Induction of EAE in rats does not produce histological features of demyelination (Brosnan et al 1988) but induces an acute mono-phasic disease pattern, unlike MS which is characterised by CNS demyelination and is in the majority of cases clinically relapsing-remitting. Chronic relapsing and demyelinating EAE models (CREAE) however are characterised by demyelination and relapse phases. With the demonstration that myelin oligodendrocyte glycoprotein (MOG) is an important neuroantigenic target in MS (Genain et al 1999) and the demonstration of far greater responses of peripheral blood auto-reactive lymphocytes to this neuroantigen, compared with MBP, in MS (Kerlero de Rosbo et al 1993, 1997) MOG induced CREAE has become the animal model of choice with features closely resembling those observed in MS (Fazakerely et al 1997, Genain et al 1999, Amor et al 1994).

Evidence from the inventor's CREAE and rat EAE feeding studies indicates that an enriched blackcurrant seed oil (72% w/w 18:3 n-6, GLA) did not protect against EAE (see Table 3). Importantly blackcurrant seed oil has a low sn-2 GLA with most of the GLA in the sn-1 and sn-3 positions (Lawson and Hughes 1988). Furthermore a structured triacylgcerol containing three GLA moieties (TG-GLA) provided protective effects similar to that of the borage oil used in CREAE (Table 2). This would also be consistent with the sn-2 GLA being important i.e. the outer pair sn-1 and sn-3 GLA being enzymatically removed in vivo and probably undergoing oxidation leaving the sn-2 GLA only. This selective hydrolysis arises from the known ability of specific lipases to remove the sn-1 and sn-3 fatty acids from triacylgycerol molecules but an apparent protection of the sn-2 position in vivo (Lawson and Hughes 1988, Kyle 1990).

This review has led the inventors to postulate that glycerides having sn-2-γ-linolenic acid, dihomo-γ-linolenic acid or arachidonic acid residues will be superior in correcting MS metabolism even to the high sn-2-γ-linolenic acid Borage oil of their earlier trial. This would allow lower doses of lipid to be taken and/or possibly decrease the time of treatment which would result in beneficial effect. U.S. Pat. No. 4,701,469 describes some potential triglycerides for nutraceutical use that the present inventors have determined may be used in the method of the invention, although it only specifically describes 1,3-dioctanyl triglycerides wherein the sn-2 acid is of an EPA, only 1,3-dioctanoyl eicosapenta glycerol is described as having been prepared. These are said to useful in inter alia immunomodulation, but although a number of diseases are specified, use in immunosuppresion in neurodegeneration and MS are not listed.

Table 3 of EP 0520624 (Efamol Holdings) compares the triglyceride content of Evening Primrose and Borage Oils, the former being taught to be more therapeutically effective than the latter for a variety of GLA responsive disorders. This document indicates Borage oil to have twenty seven different triglyceride components, only 20% of which have sn-2 GLA. Page 3, lines 40-42 notes that biological testing has shown that equal amounts of GLA may indeed have very different effects when that GLA is supplied as different oil sources. Crucially, it then directs the reader to one particular fraction present in Evening Primrose Oil (EPO), but not Borage Oil, as being responsible for the former's superior effect in raising PGE1 (see EP 0520624 Chart page 4 and Table 2) and thus anti-inflammatory effect: that fraction being identified as di-linoeoyl-mono-gamma-linolenyl-glycerol (DLMG) which it states to be 18 to 19% of the total triglyceride in EPO. Critically, page 6 clearly teaches that the position of the GLA, in sn-1, 2 or 3, is not important to this effect.

Dines et al (1994) Proceedings of the Physiological Society, Aberdeen Meeting 14-16 Sep. 1994 report on studies of treatment of diabetic neuropathy neuronal damage with γ-linolenic acid containing oils of the type advocated by EP 0520624 and again note that Borage Oil was not very effective in treating this neurodegeneration whereas Evening primmose oil was. The paper concludes that Borage Oil contains other constituents that interfere with GLA activity.

It is known that 2-arachidonyl glycerol is active in reducing TNF-α and its 1-3 median chain fatty acid triglyceride analogue has some cognitive enhancing and neurogenerative effects (see WO 01/97793 and EP 1419768). The corresponding 2-γ-linolenoyl monoglyceride is said to be inactive.

The present inventors now set out, in view of the results obtained with high sn-2-γ-linolenic acid Borage Oil, to demonstrate that it is indeed the presence of an sn-2-γ-linolenic acid, dihomo-γ-linolenic acid or arachidonic acid residue in a monoglyceride, particularly an sn-2 monoglyceride, or a metabolic precursors thereof, that gives it efficacy in treating cytokine dysregulation. Noting that the triglycerides themselves are of nearly three times the weight, and thus dose, of monoglyceride counterparts, they have determined that it is possible to administer essential fatty acids of the n-3, n-6 and n-9 type, particularly the n-6 type, as sn-2 monoglycerides and metabolic precursors thereof and still obtain beneficial cytokine changes.

The dose advantages of use of monoglycerides may be offset in part by possible increased instability of certain forms as compared with the sn-1, sn-3 saturated acyl group sn-2 EFA triglyceride exemplified in PCT/GB2004/003524. Such instability may be due e.g. to transesterification and oxidation. This issue may be addressed by producing the monoglyceride in a more stable form, e.g. a solid or semi solid rather than a liquid oil.

In a first aspect the present invention provides a method of treating a patient in need of therapy for dysregulation of cytokines TGF-β1, IL-1β, IL4, IL5, IL6, IL8, IL10, IL13 and/or γ-IFN comprising administering to that patient a therapeutically effective dose of a monoglyceride or metabolic precursor thereof of general formula I

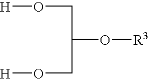

where R³ is the a fatty acyl group of an essential polyunsaturated fatty acid selected from γ-linolenoyl, γ-dihomolinolenoyl and arachidonoyl.

The present invention also provides a method for treating a patient in need of therapy for dysregulation of cytokine TNF-α comprising administering to that patient a therapeutically effective dose of a compound of general formula I wherein R³ is selected from γ-linolenoyl and γ-dihomolinolenoyl.

Particularly advantageously treated neurodegenerative diseases are those involving demyelination. The present method specifically arrests underlying neurodegeneration and restores neuronal function. Particularly the method normalises neuronal membrane composition, and restores healthy PBMC spontaneuosly released TGF-β1/TNFα ratios and the ratios of TGF-β1 with other PBMC released cytokines.

Most advantageously the method arrests neurodegeneration in multiple sclerosis of all types but particularly relapsing remitting, primary progressive and chronic progressive MS and the restoration, in part or completely, of mylein by remyleination and provides increasd neuronal function such as measured, e.g. By MRI or CAT scan or by EDSS score.

The method may also be used in treatment of cerebral impairment after stroke, head trauma and intracranial bleeding where there is demyelination or neuronal damage. Further application is provided in treating other chronic demyelination such as in Alzheimer's and Parkinson's disease.

Preferably the compound of the present invention is administered for a duration and at a dose sufficient to maintain or elevate TGF-β1 levels in the patient to therapeutic levels. By therapeutic levels is meant levels at least consistent with healthy subjects. Preferably the dose is such as to produce a TGF-β1/TNF-α ratio spontaneously released from peripheral blood mononuclear cells (PBMCs) isolated from blood of a patient, after 18 months of daily dosing, of 0.4 to 3.0, at least 0.5, more preferably at least 0.75 and most preferably at least 1. Preferably the dose is such as to produce a TGF-β1/IL-1β ratio in blood of a patient, after 18 months of daily dosing, of at least 0.5, more preferably at least 0.75 and most preferably at least 1. Preferably said levels are produced after 12 months and more preferably after 6 months.

The present invention further provides a method of treating a patient in need of remyeleination in a demyelinating disease comprising administering to that patient a therapeutically effective amount of a compound of formula I wherein R³ is selected from γ-linolenoyl and γ-dihomolinolenoyl.

For all the methods of the invention, the amount of compound administered daily will be between 0.5 and 30 grams, orally dosed, still more preferably between 0.75 and 20 grams and most preferably between 1 and 18 grams, typically 2 to 5 grams. This dose may be given as one single dose or in two or more doses together totally this amount per day.

Where the sn-2 moiety is that of a γ-linolenic acid residue, the dose may be toward the higher end of these ranges. Where the sn-2 moiety is that of a dihomo-γ-linolenic acid residue, the dose may be less, whilst where the sn-2 moiety is that of an arachidonic acid residue, efficacy is higher, but dosing should be more cautious, due to possibilities of unwanted side effects at higher levels and possible pro-inflammatory effect.

Monoglycerides for use in the invention may be accessed by known and novel (see below) methods of synthesis. For example γ-Linolenoyl glycerol (2-GLA monoglyceride, HO-GLA-OH is known (Serdarevich and Carroll, J Lipid Research, 7, 277-284 (1966); Huang et al J. Am. Oil Chem. Soc, 72, 625-631 (1995) and Aw1 et al Lipids, 24, 866-872 (1989). A variant of the known two step process uses phenylboronic acid rather than boric acid in a deprotection step and has been used for making 2-arachidonoyl glycerol but not the GLA analogue: this has now been found to be advantageous when applied to scale up.

The monoglycerides, e.g. γ-Linolenoyl glycerol, can undergo rearrangement to the 1-γ-Linolenoyl glycerol isomer when heated or treated with acids or bases. Nevertheless the 2-γ-Linolenoyl glycerol is isolatable as a yellow oil that can be stored without deterioration at −20° C. under nitrogen for several weeks at least. This exemplifies its potential for inclusion in air excluding formats such as capsules or e.g. collapsible bag containers which allow egress of oil without entry of air. A high purity process for its synthesis has also been published in Yang et al, Journal of the Chinese Institute of Chemical Engineers, 34, 617-623 (2003). These processes lend themselves to synthesis of n-3 and n-6 sn-2 monoglycerides by replacing the arachdonoyl or γ-linolenoyl source with the desired acyl group containing molecule.

A second aspect of the present invention provides compositions for use in the method of the present invention comprising the compounds of formula I together with a pharmaceutically or nutraceutically acceptable carrier, coating, capsule, diluent and/or preservative. The compounds for use in the present invention may be administered by any of the conventional vehicles known in pharmacy. Most conveniently they are administered as neat oils, optionally in solid e.g. powder forms, or in admixture with foodstuffs, in the form of capsules containing such oils, or in enterically coated forms. Other forms will occur to those skilled in the art but Remington Pharmaceutical Sciences 19^{th} Edition

By preservative is meant an antioxidant or inhibitor of transesterification. It is particularly preferred that the composition does not include Vitamin E, or includes only levels of Vitamin E that are 0.05 mg/g or less, e.g. 0.005 to 0.05 mg/g.

A third aspect of the present invention provides a pharmaceutical composition for regulating the immune system, particularly by modulating cytokines TGF-β1, IL4, IL5, IL6, IL8, IL10, IL13, and/or γ-IFN comprising a compound of general formula I as defined for the method of treatment of the invention. Compositions wherein the compounds are of formula I wherein R³ is γ-linolenoyl, γ-dihomolinolenoyl or arachidonoyl. Compositions including the compounds of formula I wherein R³ is γ-linolenoyl or γ-dihomolinolenoyl but not including the corresponding arachidonoyl compound are novel when for modulating TNF-α.

A fourth aspect of the present invention provides use of the compounds of formula I as described above for the manufacture of a medicament for the treatment of neurodegenerative diseases as set out for the method of the invention. Particularly preferred medicaments are for the arresting and reversing of neurodegeneration in multiple sclerosis of all types but particularly relapsing remitting, primary progressive and chronic progressive and the restoration, in part or completely, of neuronal integrity function such as measured, e.g. By MRI or CAT scan or by EDSS score. Other TGF-β1 responsive diseases may be treated as set out previously. Particularly treated is demyelination and remyelination is the desired result.

It will be realised by those skilled in the art that other beneficial agents may be combined with the compounds for use in the present invention or otherwise form part of a treatment regime. These might be ion channel blockers, e.g. sodium channel blockers, interferons (α, β, or γ), T-cell depleters, steroids or other palliative agents. It will further be realised that where the immune and inflammatory responses are being modulated, such combinations will need to be made carefully, given the complex nature of these systems. However, given the potential for delayed response to the present compounds, shorter acting agents might be beneficial in the first months of treatment before the cytokine levels are normalised, as long as the additional treatment does not impede this normalization process.

A fifth aspect of the present invention provides pharmaceutical compositions for the method of the invention containing, or uses of the second, third and fourth aspects of, glycerides derived from naturally occurring oils containing greater than 10% of their sn-2 fatty acids as γ-linolenoyl or γ-dihomolinolenoyl groups and that have been depleted of some or substantially all (e.g. >80%, more preferably >90% by weight) of their sn-1 and sn-3 fatty acyl groups. Such compositions include particularly triglycerides that have been treated with lipases and purified to yield compositions enriched in sn-2 monoglycerides having sn-2 γ-linolenoyl or γ-dihomolinolenoyl groups. Particularly preferred are Mucor javonicus and Borage oil derived sn-2 monoglycerides, but many other oils will occur to those skilled in the art for obtaining n-3 or n-6 enrichment, g. fish oils, blackcurrent oils, GMO canola etc (see Huang and Mills the content of which is incorporated herein).

Particularly preferred are lipase treated Borage and lipase treated High γ-linolenic acid Canola Oil.

The synthesis of compounds and compositions for use in the present invention is described below together with synthesis of comparative examples. The synthesis is exemplified by reference to oils containing sn-2 γ-linolenoyl groups. The corresponding dihomo-γ-linolenoyl and arachidonoyl compounds are provided by use of the corresponding starting materials.

The present invention will now be described by way of Example only by reference to the following non-limiting Tables, Examples and Figures. Further embodiments falling within the scope of the invention will occur to those skilled in the art in the light of these.

### FIGURES

FIG. 1: Shows spontaneous peripheral blood mononuclear cell cytokine production in placebo and high sn-2 γ-linolenic acid, PCT/GB04/002089 trial oil treated human MS patients at 18 months. Left column is placebo and right treatment in each case.

FIG. 2: Shows the effect of placebo and low dose (5 g/day) high sn-2 GLA Borage oil on human MS patient EDSS score as compared to high dose (15 g/day) displayed as a histogram with months treatment on the x axis.

FIG. 3: Shows the effect of placebo, low dose and high dose high sn-2 GLA Borage oil on human MS patient Mean Relapse rate (%) as histogram with months on x axis.

FIG. 4: Shows the reaction scheme for preparation of CGC: intermediate for the preparation of monoglycerides for use in the method of the present invention.

FIG. 5: Shows the reaction scheme for preparation of glycerol 2-octa-6Z,9Z,12Z-trienoate (a compound for use in the method of the invention) through an intermediate and novel metabolic precursor compound for use in the method of the invention 1,3-O-benzylidene glycerol 2-octa-6Z,9Z,12Z-trienoate.

FIG. 6: shows the reaction scheme for production of 2-GLA MG (γ-linolenic acid monoglyceride) from CGC (Glycerol 1,3-didecanoate-2-γ-linolenoate) and Borage oil using lipases.

### EXAMPLES

### Background

High sn-2 Borage Oil (PCT/GB04/002089) Trial.

### Isolation and Culture of PBMC

Heparinised whole blood was diluted with an equal volume of Hanks' balanced salt solution (Sigma, UK) and the resulting diluted blood layered onto Lymphoprep (Nycomed, Oslo, Norway). Following density centrifugation at 800 g for 30 minutes the PBMC were removed from the interface and diluted in Hanks' solution. The cells were then washed twice by centrifugation for 10 minutes at 250 g. The resulting final pellet was then resuspended in culture medium consisting of RPMI-1640 medium (Sigma, UK) supplemented with 2 mM L-glutamine, 100 U penicillin and 100 μg streptomycin (Sigma, UK) and 10% autologous plasma. 2×10⁶ per ml PBMC, >95% viable as judged by trypan blue exclusion, were added to tissue culture tubes (Bibby Sterilin Ltd, Stone, UK) and incubated for 24 h at 37° C. with 5% CO₂. The concentration of antigen, cell density and time of culture were all determined in previous kinetic experiments to determine maximum cytokine production (data not shown). Routine cytospin preparations were also prepared for subsequent differential counts. Following incubation the cells were removed from culture by centrifugation at 250 g for 10 minutes, the resulting supernatants were then removed, aliquoted and stored at −70° C.

### Preparation of Plasma Samples

10 ml of heparinised blood was spun at 250 g for 10 minutes. The resulting plasma layer was then removed, aliquoted and stored at −70° C.

### Detection of Pro-Inflammatory Cytokines

TNF-α, IL-1β and IFN-γ in cell culture supernatants and plasma were detected using commercially available paired antibodies enabling cytokine detection in an ELISA format (R&D systems Ltd, Abingdon, UK). The sensitivities for the TNF-α and IFN-γ ELISAs were 15.6-1000 pg/ml and 3.9-250 pg/ml for IL-1β.

### Detection of Biologically Active TGF-β1

Biologically active TGF-β1 in cell culture supernatants and plasma were detected using the commercially available Eₘₐₓ ELISA system with a sensitivity of 15.6-1000 pg/ml (Promega, Southampton, UK).

### Statistical Analysis

Differences in cytokine production were compared using Student's t-test and Mann-Whitney U-test and were considered significant when p values were less than 0.05.

### Results

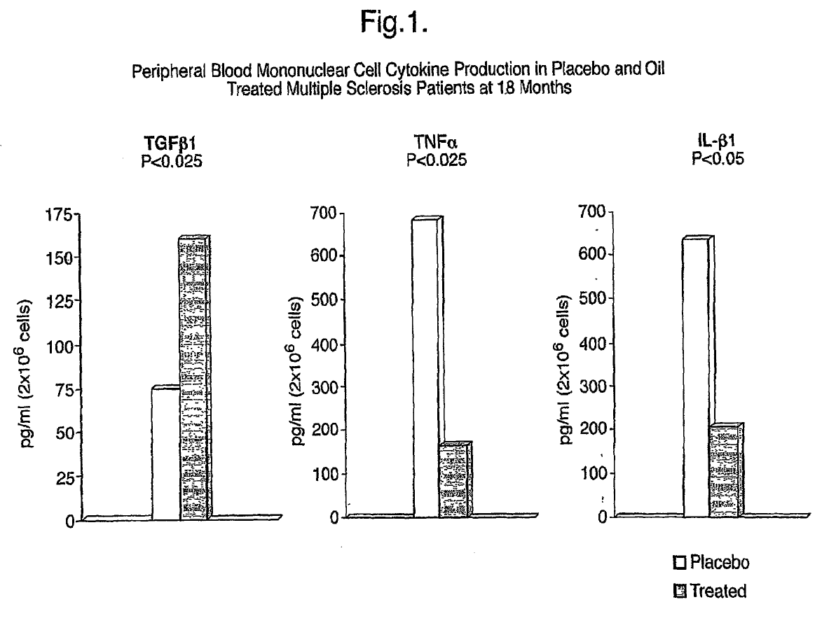

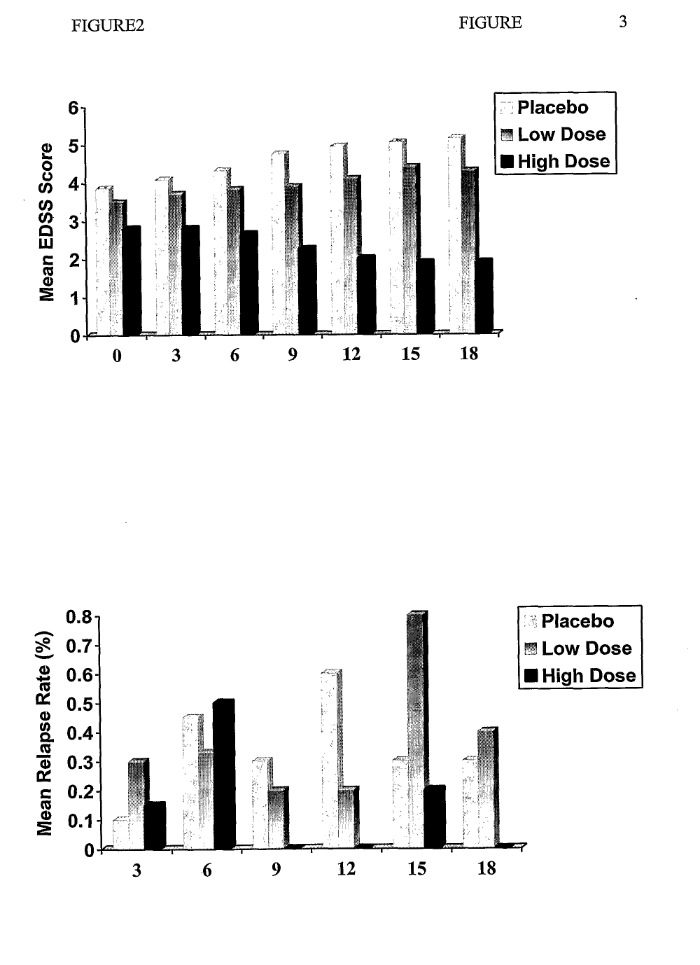

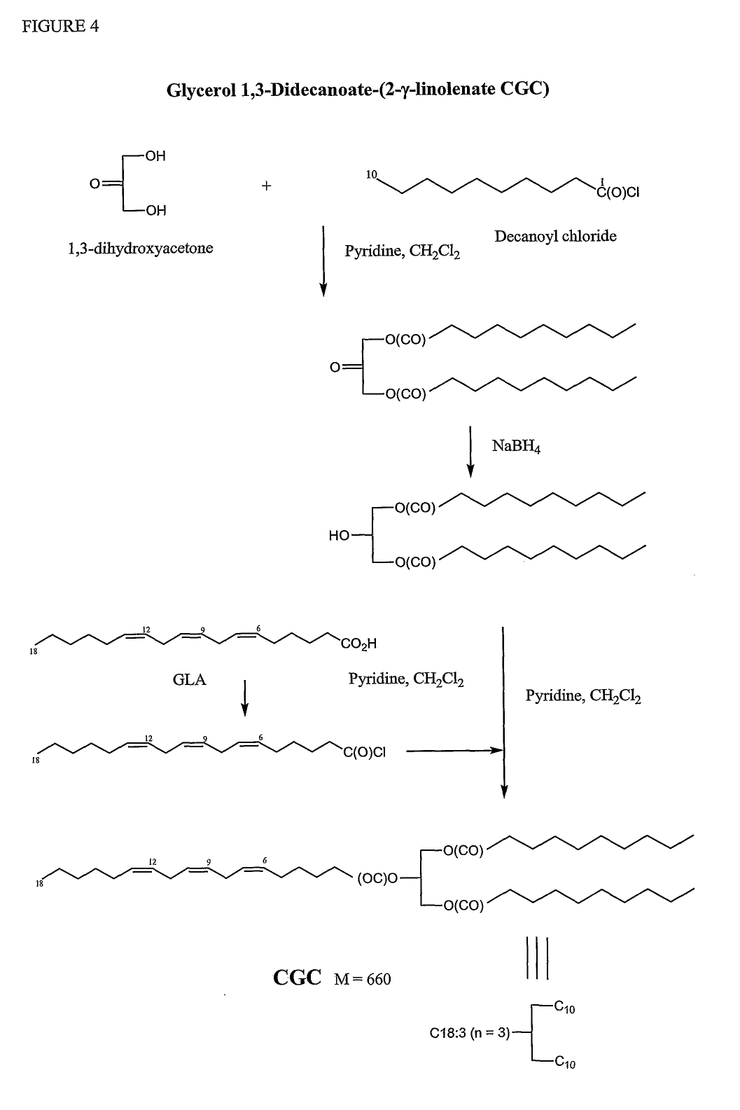

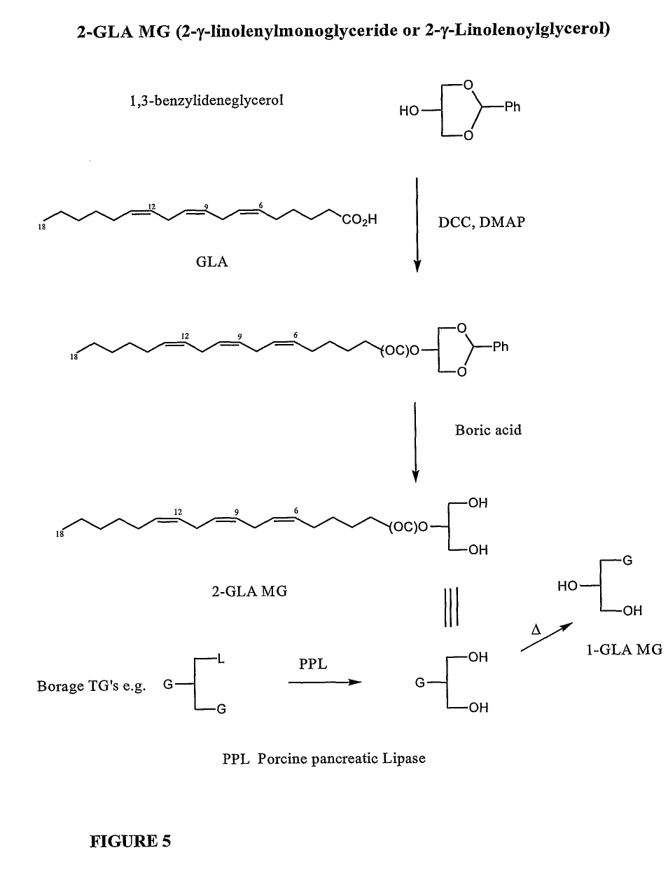

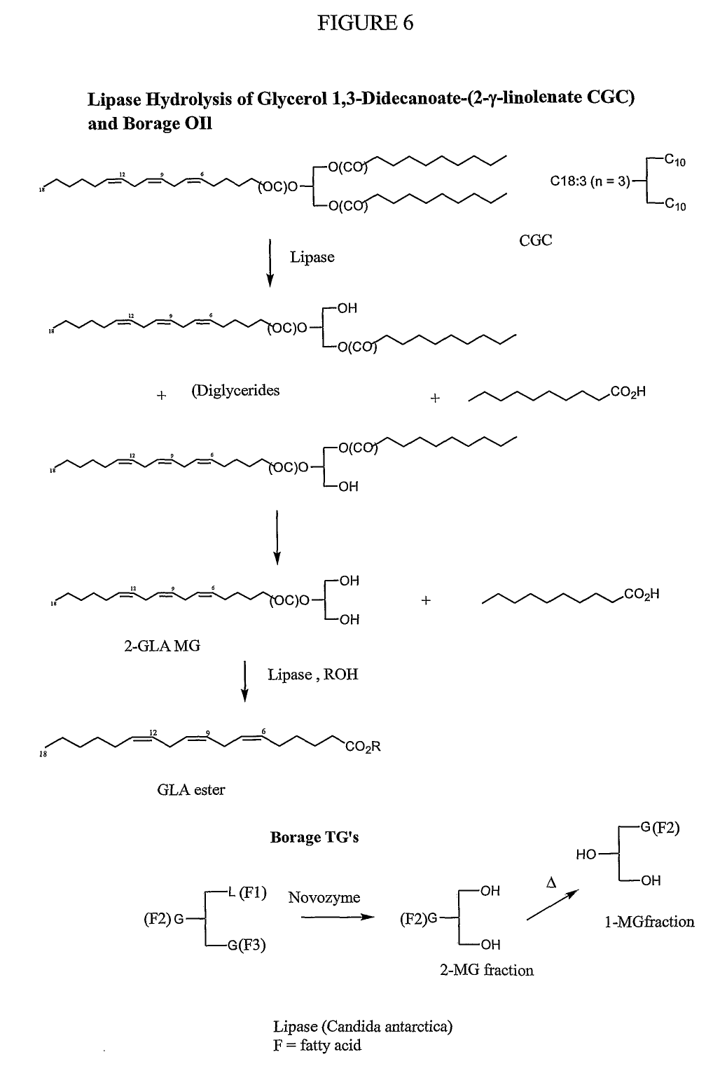

See FIG. 1

### Experimental Procedure

The proton-decoupled ¹³C NMR spectra with suppressed NOE were collected at 21° C. in a 5-mm broadband probe on a Joel 500 MHz spectrometer operating at 125.728 MHz. Waltz decoupling was the chosen mode of decoupling and was gated on only during the 14.89 s acquisition time. The relaxation delay was set at 30 secs and the pulse angle was 900. The spectral window used was ca. 35 ppm (from 173.5 to 172.6 ppm) with a 170 ppm offset. The spectra were internally referenced to CDCl₃ at 77.0 ppm. Typically, the approximate number of scans collected for adequate signal-to-noise ranged from 300 to 1200 scans depending on the concentration and purity of the sample. The total acquisition time for the experiments ranged between 2-8 h e.g. 1272 scans; data points 65,536. Concentrated solutions up to 20% w/v were employed when possible to reduce the acquisition time The chemical shifts quoted vary with the concentration of the solution.

### Synthesis of Compounds for Use in the Present Invention.

### Example 1

### 2-γ-linolenoyl glycerol

### glycerol 2-octa-6Z,9Z,12Z-trienoate) HO-GLA-OH

### 1a) Preparation of 1,3-O-benzylidene glycerol 2-octa-6Z,9Z,12Z-trienoate

### Intermediate and Compound of the Invention where R4 is H and R5 is Benzylidene

Oxaloyl chloride (7.8 ml, 11.3 g, 0.089 mol, 0.95 equivalents) was added over 2-3 minutes to a stirred solution of γ-linolenic acid (GLA95, 16.7 g, 0.060 mol, 0.64 equivalents-Scotia) in dichloromethane (100 ml) under N₂. The mixture was stirred overnight at room temperature and then concentrated in vacuo to give a tan oil. This crude γ-linolenoyl chloride was added over ca 10 minutes to a stirred solution of 1,3-O-benzylidene glycerol (13.0 g, 0.094 mol, 1 equivalent), dry pyridine (30 ml, 29.3 g, 0.37 mol, 4 equivalents) and dichloromethane (DCM, 120 ml) at 5° C. and the mixture then stirred at room temperature for 2 hours. The reaction mixture was filtered and then the filtrate washed with DCM. The combined filtrate and washings were then washed with water (2×20 ml) and the DCM extract dried over Mg SO4 and concentrated in vacuo to give a crude product as a tan oil (purity>90% by HPLC). The oil was purified by column chromatography on silica gel (300 g). Elution with DCM gave the product as a yellow oil (19.2 g (73%), 96.3% purity by HPLC).

### 1b) Preparation of glycerol 2-octa-6Z,9Z,12Z-trienoate

Boric acid (2.81) g, 0.045 mol, 4 equivalents) was added to a freshly prepared solution of 1,3-O-benzylidene glycerol 2-octa-6Z,9Z,12Z-trienoate (5.0 g, 0.011 mol, 1 equivalent) in trimethyl borate (28 ml, 26.0 g, 0.25 mol, 22 equivalents) under N2. The mixture was stirred and heated in a sealed Teflon bomb at 90° C. for 15 minutes. The resulting cool solution was concentrated in vacuo and the residue heated at 90° C. under vacuum for 5 minutes. HPLC analysis indicated that 96% of the starting acetal had reacted. This process was repeated on the same scale twice more and the combined residues were dissolved in ether (150 ml) and washed with water (2×50 ml). The ether extract (not dried as MgSO₄ promotes rearrangement) was concentrated in vacuo, ethanol added to the residue and the solution re-evaporated with ethanol (2×) to remove water. The residual tan oil (15.0 g) was purified by column chromatography on solica gel-boric acid (10:1) (10 g). Elution with DCM and then DCM-methanol (9:1) gave the product as a yellow oil (11.7 g, 73%) HPLC purity 93.4%. This material was stored in a freezer at −20° C. under N₂. Little if any deterioration was observed over 21 days.

δ_{H} (500 MHz, CDCl₃) 0.89 (3H, t, J=7.0 Hz, C—CH₃), 1.25-1.45 (8H, complex m, 4×CH₂), 1.66 (2H, p, J=7.4 Hz, CH₂—C—CO), 2.06 (2H, m, 2×CH₂C═C), 2.37 (2H, m, CH₂CO), 2.81 (2×C═CCH₂C═C), 3.79 (4H, d, J=4.5 Hz, 2×OCH₂), 4.90 (1H, p, J=4.7 Hz, OCH), 5.36 (6H, m, 3×C═C).

δ_{C} (125.7 MHz, CDCl₃) 14.1 (CH₃), 22.59, 24.54, 25.65, 26.86, 27.22, 29.06, 29.33, 31.52, 34.21, 61.95 (OCH₂), 74.87 (OCH), [127.59, 128.02, 128.35, 128.45, 129.49, 130.44, olefinic carbon], 173.85 carbonyl.

### Example 2

### Production of Monoglyceride Enriched Compositions from Synthetic CGC Structured-Lipid

### 1. 2-GLA MG (γ-Linolenic Acid Monoglyceride) from CGC (Glycerol 1,3-didecanoate-2-γ-linolenoate)

Lipase acrylic resin from Candida antarctica (Sigma, Novozyme, 0.1 g) was added to a solution of CGC (0.25 g) in ethanol (0.75 ml) prepared as described. The mixture was stirred at 35-40° C. and monitored by HPLC. After 3 h the resin was removed by filtration and washed with ethanol. The filtrate and washings were concentrated in vacuo. Analysis of the residual oil by HPLC indicated the formation of two major products. These were separated by chromatography on silica-boric acid. Elution with dichloromethane (DCM) gave an oil (fraction A, 160 mg). Further elution with DCM-MeOH (9:1) gave an oil (fraction B, 80 mg). HPLC comparison with authentic materials indicated that B was the required product i.e. 2-GLA MG (8% rearranged 1-isomer also present). The main (>90%) component of fraction A was found (by HPLC comparison and NMR) to be ethyl decanoate. The minor component was found (by HPLC and NMR) to be ethyl γ-linolenoate. These esters are expected to be formed under the reaction conditions from the corresponding acids (C and GLA) and ethanol.

### Example 3

### Production of 2-GLA Monoglyceride Containing Fraction from Borage Oil

### Containing Triglycerides XGX

Lipase acrylic resin from Candida antarctica (Sigma, Novozyme, 0.50 g) was added to a solution of borage oil (1.25 g) in ethanol (4 ml). The mixture was stirred at 35-40° C. and monitored by HPLC. After 3 h the resin was removed by filtration and washed with ethanol. The filtrate and washings were concentrated in vacuo. Analysis of the residual oil (1.19 g) by HPLC indicated the formation of several products. These were partially separated by chromatography on silica-boric acid (40 g). Elution with dichloromethane (DCM, 400 ml) gave an oil (fraction A, 0.85 g). Further elution with DCM-MeOH (9:1) gave an oil (fraction B, 0.20 g). HPLC comparison with authentic materials indicated that B contained 2-GLA MG plus 8% rearranged 1-isomer along with other monoglycerides. These monoglycerides are believed to be predominantly PUFAs (polyunsaturated fatty acid) α-linolenic, linoleic acid and a minor amount of stearadonic acid (an n-3 EFA precursor). Several minor peaks were observed by HPLC. Monoglycerides containing saturated fatty acids or mono-ene fatty acids may not be detected by HPLC under these conditions. (reverse phase C18, acetonitrile-isopropanol gradient, 2 ml/min, UV detector 210 nm)

### Biological Studies.

### Example 4

Solublization of Sn-2 monoglyceride was performed using ethyl alcohol or DMSO for in vitro work on human peripheral blood mononuclear cells (PBMCs). A tendency to precipitate at acid pH may have been the cause of some animals regurgitating solid material after gavage suggesting that enterically coated formulation may be preferred. SJL mice were fed sn-2 GLA of Example 1 at three doses (50, 125 and 250 μl) for seven days by gavage. Mice receiving higher doses were prone to regurgitation. After seven days animals were killed and the brain, liver and spleen were removed—the liver and brain frozen at −70° C. and mononuclear cells were isolated from spleens by sieving and density centrifugation on Lymphoprep (Sigma Chemical Co) and cultured at 37° C. in 5% CO2 atmosphere in 5 ml culture tubesat a cell density of 1×10⁶ cells/ml in RPMI 1640 medium in 5% foetal calf serum (FCS). Cells were cultured with and without 1 μg/ml or 25 μg/ml concanavalin A (Con A) for approximately 20 hours and the supernatants removed and stored at −70° C. until required. Mouse TGF-β1 was measured in supernatants using a commercially available ELISA (Promega, Madison Wis.).

TABLE 1
Stimulated (Con A) and unstimulated TGF-β1 production pg/ml from
spleen PBMCs in response to feeding of sn-2-γ-linolenoyl-glycerol
monoglyceride of Example 1b

| | Con A (μg/ml | | |
|---|---|---|---|
| Monoglyceride | 0 | 1 | 25 |
| 50 μg | 377 | 409 | 480 |
| Control | 209 | 228 | 393 |
| Change % | 80 | 79 | 42 |

### REFERENCES

Amor S, Groome N, Linington C, Morris M M, Dornmair K, Gardinier M V, Matthieu J M, Baker D. Identification of epitopes of myelin oligodendrocyte glycoprotein for the induction of experimental allergic encephalomyelitis in SJL and Biozzi AB/H mice. J Immunol. 1994 Nov. 15; 153(10):4349-56.
Beck J, Rondot P, Catinot L et al. Increased production of interferon gamma and tumor necrosis factor precedes clinical manifestation in multiple sclerosis: do cytokines trigger off exacerbations? Acta Neurol Scand 1988; 78:318-23.
Bertolotto A, Capobianco M, Malucchi S et al. Transforming growth factor beta1 (TGFbeta1) mRNA level correlates with magnetic resonance imaging disease activity in multiple sclerosis patients. Neurosci Lett 1999; 263:21-4.
Bertolotto A, Malucchi S, Capobianco M et al. Quantitative PCR reveals increased levels of tumor necrosis factor-alpha mRNA in peripheral blood mononuclear cells of multiple sclerosis patients during relapses. J Interferon Cytokine Res 1999; 19:575-81.
Brosnan C F., Selmaj K and Raine C S. Hypothesis: a role for tumor necrosis factor in immune-mediated demyelination and its relevance to multiple sclerosis. J Neuroimmunol 1988:18, 87-94.
Brosnan C F and Raine C S. Mechanisms of immune injury in multiple sclerosis. Brain Pathol. 1996:6, 243-257.
Burns J, Bartholomew B, Lobo S. Isolation of myelin basic protein-specific T cells predominantly from the memory T-cell compartment in multiple sclerosis. Ann Neurol 1999; 45:33-9.
Cannella B, Raine C S. The adhesion molecule and cytokine profile of multiple sclerosis lesions. Ann Neurol 1995; 37:424-35.
Chou Y K, Bourdette D N, Offner H et al. Frequency of T cells specific for myelin basic protein and myelin proteolipid protein in blood and cerebrospinal fluid in multiple sclerosis. J Neuroimmunol 1992; 38:105-14.
De Stefano N., Narayanan S., Francis G S., Arnaoutelis R., Tartaglia M C., Antel J P., Matthews P M and Arnold D L. Evidence of axonal damage in the early stages of multiple sclerosis and its relevance to disability. Arch Neurol. 2001: 58(1), 65-70.
Ewing C, Bernard C C. Insights into the aetiology and pathogenesis of multiple sclerosis. Immunol Cell Biol 1998; 76:47-54.
Fazakerly J K. Molecular biology of multiple sclerosis. Wiley and Sons Ltd. 1997, 255-273.
Fredrikson S, Soderstrom M, Hillert J et al. Multiple sclerosis: occurrence of myelin basic protein peptide-reactive T cells in healthy family members. Acta Neurol Scand 1994; 89:184-9.
Genain C P., Cannella B., Hauser S L and Raine C S. Identification of autoantibodies associated with myelin damage in multiple sclerosis. Nature Med 1999:5, 170-175.
Gross C E, Bednar M M, Howard D B and Spom M B (1993) Transforming growth factor beta I reduces infarct size after experimental cerebral ischemia in a rabbit model Stroke 24, 558-562.
Harbige, L S, Crawford M A, Jones J, Preece A W and Forti A. Dietary intervention studies on the phosphoglyceride fatty acids and electrophoreitic mobility of erythrocytes in multiple sclerosis. Prog. Lipid Res 1986:25, 243-248.
Harbige L S. Nutrition and immunity with emphasis on infection and autoimmune disease. (1996) Nutr Health, 10(4):285-312.
Harbige L S (1998) Dietary n-6 and n-3 fatty acids in immunity and autoimmune disease. Proceedings of the Nutrition Society 57, 555-562.
Harbige L S, Yeatman N, Amor S & Crawford M A (1995) Prevention of experimental autoimmune encephalomyelitis in Lewis rats by a novel source of y-linolenic acid. British Journal of Nutrition 74, 701-715.
Harbige L S., Layward L., Morris-Downes M M., Dumonde D C and Amor S. The protective effects of omega-6 fatty acids in experimental autoimmune encephalomyelitis (EAE) in relation to transforming growth factor-beta 1 (TGF-beta1) up-regulation and increased prostaglandin E2 (PGE2) production. Clin Exp Immunol 2000:122, 445-452.
Henrich Noack P, Prehn J H, and Kriegistein J. (1996) TGF-beta I protects hippocampal neurons against degeneration caused by transient global ischaemia. Dose-response relationship and potential neuroprotective mechanisms. Stroke, 27, 1609-1614.

Hirsch R L, Panitch H S, Johnson K P. Lymphocytes from multiple sclerosis patients produce elevated levels of gamma interferon in vitro. J Clin Immunol 1985; 5:386-9.
Hollifield R D, Harbige L S, PhM-Dinh D, Sharief M. Evidence for cytokine Dysregulation in Multiple Sclerosis: Peripheral Blood Mononuclear cell production of pro-inflammatory and anti-inflammatory cytokines during relapse and remission. Autoimmunity, 2003 36(3):133-141.
Huang and Mills (Eds) 1996 Gamma-linolenic acid metabolism and its roles in nutrition and medicine. AOCS Press, Champagne, Ill., 319 pp.
Imamura K, Suzumura A, Hayashi F et al. Cytokine production by peripheral blood monocytes/macrophages in multiple sclerosis patients. Acta Neurol Scand 1993; 87:281-5.
Issazadeh S, Lorentzen J C, Mustafa M I et al. Cytokines in relapsing experimental autoimmune encephalomyelitis in DA rats: persistent mRNA expression of proinflammatory cytokines and absent expression of interleukin-10 and transforming growth factor-beta. J Neuroimmunol 1996; 69:103-15.
Johns L D, Sriram S. Experimental allergic encephalomyelitis: neutralizing antibody to TGF beta 1 enhances the clinical severity of the disease. J Neuroimmunol 1993; 47:1-7.
Kerlero de Rosbo N, Hoffman M, Mendel I et al. Predominance of the autoimmune response to myelin oligodendrocyte glycoprotein (MOG) in multiple sclerosis: reactivity to the extracellular domain of MOG is directed against three main regions. Eur J Immunol 1997; 27:3059-69.
Kerlero de Rosbo N, Milo R, Lees M B et al. Reactivity to myelin antigens in multiple sclerosis. Peripheral blood lymphocytes respond predominantly to myelin oligodendrocyte glycoprotein. J Clin Invest 1993; 92:2602-8.
Khalil N. TGF-beta: from latent to active. Microbes Infect 1999; 1:1255-63.
Krupinski J, Kumar P, Kumar S, and Kaluza J. (1996) Increased expression of TGF-beta I in brain tissue after ischemic stroke in humans. Stroke, 27, 852-857.
Kuroda Y, Shimamoto Y. Human tumor necrosis factor-alpha augments experimental allergic encephalomyelitis in rats. J Neuroimmunol 1991; 34:159-64.
Lu C Z, Jensen M A, Arnason B G. Interferon gamma- and interleukin-4-secreting cells in multiple sclerosis. J Neuroimmunol 1993; 46:123-8.
Maimone D, Reder A T, Gregory S. T cell lymphokine-induced secretion of cytokines by monocytes from patients with multiple sclerosis. Cell Immunol 1993; 146:96-106.
Martino G, Hartung H-P. Immunopathogenesis of multiple sclerosis: the role of T cells. Curr Opin Neurol 1999; 12:309-21.
McCarron R M, Wang L, Racke M K et al. Cytokine-regulated adhesion between encephalitogenic T lymphocytes and cerebrovascular endothelial cells. J Neuroimmunol 1993; 43:23-30.
McDonald W I, Compston A, Edan G, Goodkin D, Hartung H P, Lublin F D, McFarland H F, Paty D W, Polman C H, Reingold S C, Sandberg-Wollheim M, Sibley W, Thompson A, van den Noort S, Weinshenker B Y, Wolinsky J S. Recommended diagnostic criteria for multiple sclerosis: guidelines from the International Panel on the diagnosis of multiple sclerosis. Ann Neurol. 2001 July; 50(1):121-7.
Merrill J E, Strom S R, Ellison G W et al. In vitro study of mediators of inflammation in multiple sclerosis. J Clin Immunol 1989; 9:84-96.
Merrill J E, Zimmerman R P. Natural and induced cytotoxicity of oligodendrocytes by microglia is inhibitable by TGF beta. Glia 1991; 4:327-31.
Miyazono K, Hellman U, Wernstedt C et al. Latent high molecular weight complex of transforming growth factor beta 1. Purification from human platelets and structural characterization. J Biol Chem 1988; 263:6407-15.
Mokhtarian F, Shi Y, Shirazian D et al. Defective production of anti-inflammatory cytokine, TGF-beta by T cell lines of patients with active multiple sclerosis. J Immunol 1994; 152:6003-10.
Navikas V, Link H. Review: cytokines and the pathogenesis of multiple sclerosis. J Neurosci Res 1996; 45:322-33.
Noseworthy J H. Progress in determining the causes and treatment of multiple sclerosis. Nature 1999:399(6738 Suppl), A40-47.
Ota K, Matsui M, Milford E L et al. T-cell recognition of an immunodominant myelin basic protein epitope in multiple sclerosis. Nature 1990; 346:183-7.
Perkin G D, Wolinsky J S. Fast facts-Multiple Sclerosis, 1st Edn. Oxford, UK: Health Press, 2000.
Philippe J, Debruyne J, Leroux-Roels G et al. In vitro TNF-alpha, IL-2 and IFN-gamma production as markers of relapses in multiple sclerosis. Clin Neurol Neurosurg 1996; 98:286-90.
Phylactos A C, Ghebremeskel K, Costeloe K, Leaf A A, Harbige L S, Crawford M A. (1994) Polyunsaturated fatty acids and antioxidants in early development. Possible prevention of oxygen-induced disorders. Eur J Clin Nutr. 48 Suppl 2:S17-23.
Prehn J H, Peruche B, Unsicker K and Kriegistein J. (1993) Isoform-specific effects of transforming growth factor-beta on degeneration of primary neuronal cultures induced by cytotoxic hypoxia or glutamate. J. Neurochem. 60, 1665-1672.
Rack M K, Sriram S, Calrimi J, Cannella B, Raine C S & McFarim D E (1993) Long-term treatment of chronic relapsing experimental allergic encephalomyelitis by transforming growth factor-p2. Journal of Neuroimmunology, 46, 175-183.
Racke M K, Cannella B, Albert P et al. Evidence of endogenous regulatory function of transforming growth factor-beta 1 in experimental allergic encephalomyelitis. Int Immunol 1992; 4:615-20.
Rieckmann P, Albrecht M, Kitze B et al. Cytokine mRNA levels in mononuclear blood cells from patients with multiple sclerosis. Neurology 1994; 44:1523-6.
Rieckmann P, Albrecht M, Kitze B et al. Tumor necrosis factor-alpha messenger RNA expression in patients with relapsing-remitting multiple sclerosis is associated with disease activity. Ann Neurol 1995; 37:82-8.
Ruddle N H, Bergman C M, McGrath K M et al. An antibody to lymphotoxin and tumor necrosis factor prevents transfer of experimental allergic encephalomyelitis. 3 Exp Med 1990; 172:1193-200.
Santambrogio L, Hochwald G M, Saxena B, Leu C H, Martz J E, Carlino J A, Ruddle N H, Palladino M A, Gold L I & Thorbecke G J (1993) Studies on the mechanisms by which Transforming Growth Factor-p protects against allergic encephalomyelitis. Journal of Immunology 151, 1116-1127.
Schiefer H B, Hancock D S, Loew F M. Long-term effects of partially hydrogenated herring oil on the rat myocardium. Drug Nutr Interact. 1982; 1(2):89-102.
Schluesener H J, Lider O. Transforming growth factors beta 1 and beta 2: cytokines with identical immunosuppressive effects and a potential role in the regulation of autoimmune T cell function. J Neuroimmunol 1989; 24:249-58.
Selmaj K, Raine C S, Cannella B et al. Identification of lymphotoxin and tumor necrosis factor in multiple sclerosis lesions. J Clin Invest 1991; 87:949-54.
Selmaj K, Raine C S, Farooq M et al. Cytokine cytotoxicity against oligodendrocytes. Apoptosis induced by lymphotoxin. J Immunol 1991; 147:1522-9.
Sharief M K, Thompson E J. In vivo relationship of tumor necrosis factor-alpha to blood-brain barrier damage in patients with active multiple sclerosis. J Neuroimmunol 1992; 38:27-33.
Tejada-Simon M V, Hong J, Rivera V M et al. Reactivity pattern and cytokine profile of T cells primed by myelin peptides in multiple sclerosis and healthy individuals. Eur J Immunol 2001; 31:907-17.
Vartanian T, Li Y, Zhao M et al. Interferon-gamma-induced oligodendrocyte cell death: implications for the pathogenesis of multiple sclerosis. Mol Med 1995; 1:732-43.
Vivien D, Bemaudin M, Buisson A, Divoux D, MacKenzie E T and Nouvelot A. (1998) Evidence of type I and type II transforming growth factor-beta receptors in central nervous tissues: changes induced by focal cerebral ischemia. J. Neurochem. 70, 2296-2304.
Zhang J, Markovic-Plese S, Lacet B et al. Increased frequency of interleukin 2-responsive T cells specific for myelin basic protein and proteolipid protein in peripheral blood and cerebrospinal fluid of patients with multiple sclerosis. J Exp Med 1994; 179:973-84.
Japanese Patent 6172263 (1994) Y. Kosugi et al, Agency of Industrial Science & Technology High-purity arachidonic acid triglyceride and its production.
U.S. Pat. No. 4,888,324 (1989) N. Catsimpoolas et al, Angio-Medical Corporation Method for enhancing angiogenesis with lipid containing molecules.
Y. Kosugi and N. Azuma, J. Amer. Oil Chem. Soc., 71, 1397-1403 (1994). Synthesis of Triacylglycerol from polyunsaturated fatty acid by immobilized ipase.
J. W. Hageman et al, J. Amer, Oil Chem. Soc., 49, 118-xxx (1972) Preparation of Glycerin and their Uses.
E. S. Lutton and A. J. Fehl, Lipids, 5, 90-99 (1970). The polymorphism of odd and even saturated single acid triglycerides, C8-C22.
D. Horrobin, A. McMordie, M. S. Manku (Scotia Holdings PLC UK) Eur. Pat. Appl EP 609078 3 Aug. 1994. Phospholipids containing two different unsaturated fatty acids for use in therapy, nutrition, and cosmetics.
Y.-S. Huang, X. Lin, P. R. Redden and D. F. Horrobin, J. Am. Oil Chem. Soc., 72, 625-631, (1995). In vitro Hydrolysis of Natural and Synthetic γ-Linolenic Acid-Containing Triacylglycerols by Pancreatic Lipase
K. Osada, K. Takahashi, M. Hatano and M. Hosokawa, Nippon Suisan Gakkaishi., 57, 119-125 (1991). Chem. Abstr., 115:278299 Molecular Species of Enzymically-synthesized Polyunsaturated Fatty acid-rich Triglycerides.
J.-W. Liu, S. DeMichele, M. Bergana, E. Bobik, Jr., C. Hastilow, Lu-Te Chuang, P. Mukeiji and J.-S. Huang., J. Am. Oil Chem. Soc., 78, 489-493 (2001) Characterization of Oil Exhibiting High γ-Linolenic Acid from a Genetically transformed Canola Strain.
D. R. Kodali, D. Atkinson, T. G. Redgrave and D. Small, J. Lipid Res., 28, 403-413 (1987). Structure and polymorphism of 18-Carbon Fatty Acid Triacylglycerols: Effect of Unsaturation and Substitution in the 2-Position
P. H. Bentley and W. McCrae, J. Org. Chem. 35, 2082-2083 (1970) An Efficient Synthesis of Symmetrical 1,3-Diglycerides.
M. Berger, K. Laumen and M. P. Schneider, J. Am. Oil. Chem. Soc., 69, 955-959, (1992). Enzymatic Esterification of Glycerol 1. Lipase-Catalyzed Synthesis of Regioisomerically Pure 1,3-sn-Diacylglycerols.
A. P. J. Mank, J. P. Ward and D. A. van Dorp, Chem. Physics Lipids, 16, 107-114 (1976). A versatile, flexible synthesis of 1,3-diglycerides and triglycerides.
L. Hartman, Chem. Rev., 58, 845-867 (1958) and references therein. Advances in the Synthesis of Glycerides of Fatty Acids

1. A method of treating a patient in need of therapy for dysregulation of TGF-β1, IL-1β, IL4, IL5, IL6, IL8, IL10, IL13 and/or γ-IFN comprising administering to that patient a therapeutically effective dose of a monoglyceride of general formula I 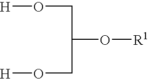
wherein R¹ is the fatty acyl group of an essential polyunsaturated fatty acid selected from γ-linolenoyl, γ-dihomolinolenoyl and arachidonoyl.

2. A method as claimed in claim 1 wherein the patient is in need of therapy for a neurodegenerative diseases which involves demyelination.

3. A method as claimed in claim 2 wherein the therapy results in remyelination.

4. A method as claimed in claim 1 wherein the therapy decreases EDSS score.

5. A method as claimed in claim 1 wherein the patient is in need of therapy for multiple sclerosis.

6. A composition for use in the method of the present invention comprising a compound of formula I together with a pharmaceutically or nutraceutically acceptable carrier, coating, capsule, diluent and/or preservative.

7. A composition as claimed in claim 17 comprising a preservative which is an antioxidant or inhibitor of transesterification.

8. A method as claimed in claim 7 wherein the preservative or inhibitor comprises 0.05 mg/g or less of Vitamin E.

9. A pharmaceutical composition for regulating the immune system comprising a compound of general formula I as defined in claim 1.

10. Use of a compound of formula I as described in claim 1 for the manufacture of a medicament for the treatment of dysregulation of cytokines TGF-β1, IL-1, IL4, IL5, IL6, IL8, IL10, IL13 and/or γ-IFN or for the modulation of these cytokines.

11. Use as claimed in claim 10 wherein the use is for manufacture of a medicament for treating neurodegenerative diseases.

12. Use as claimed in claim 10 wherein the medicament is for the arresting and reversing of neurodegeneration in multiple sclerosis of all types but particularly relapsing remitting, primary progressive and chronic progressive and the restoration, in part or completely, of neuronal integrity function such as measured, e.g. By MRI or CAT scan or by EDSS score.

13. A method as claimed in claim 1 wherein the compound of formula I is in the form of a glyceride containing composition containing greater than 10% of its sn-2 fatty acids as γ-linolenoyl, γ-dihomolinolenoyl and arachidonoyl and lacking of some or substantially all (e.g. >80%, more preferably >90% by weight) of the glyceride sn-1 and sn-3 fatty acyl groups.

14. A method or use as claimed in claim 13 wherein the glyceride is derived from a triglyceride that has been treated with lipases and purified to yield compositions enriched in sn-2 monoglycerides.

15. A method or use as claimed in claim 14 wherein the triglyceride is a Mucor javonicus, Borage oil, fish oils, black current oils, evening primrose oil or GMO canola oil that has been depleted of some or substantially all of its sn-1 and sn-3 fatty acyl groups.

16. A method as claimed in claim 1 wherein the neurodegenerative disease involves demyelination.

17. A method as claimed in claim 1 wherein the treatment specifically arrests underlying neurodegeneration and restores neuronal function.

18. A method as claimed in claim 1 which normalises neuronal membrane composition with respect to γ-linolenic acid, dihomo-γ-linolenic acid and arachidonic acid lipid content.

19. A method as claimed in claim 1 which restores healthy TGF-β1/TNFα ratios as measured from spontaneous release from peripheral blood mononuclear cell release.

20. A method as claimed in claim 1 wherein the disease is relapsing remitting multiple sclerosis, primary progressive multiple sclerosis or chronic progressive multiple sclerosis.

21. A method as claimed in claim 1 wherein the treatment is of cerebral impairment after stroke, head trauma and intracranial bleeding, Alzheimer's disease or Parkinson's disease where there is demyelination or neuronal damage.

22. A method as claimed in claim 1 wherein the lipid is administered for a duration and at a dose sufficient to maintain or elevate TGF-β1 levels in the patient to therapeutic levels.

23. A method as claimed in claim 1 wherein the lipid is administered for a duration and at a dose sufficient to maintain or elevate TGF-β1 levels in the patient to a TGF-β1/TNF-α ratio released spontaneously from peripheral blood mononuclear cells isolated from the blood of a patient, after 18 months of daily dosing, of 0.4 to 3.0, at least 0.5 more preferably at least 0.75 and most preferably at least 1.

24. A method as claimed in claim 1 wherein the dose is such as to produce a TGF-β1/IL-1β ratio in PBMCs isolated from blood of a patient, after 18 months of daily dosing, of at least of at least 0.75.

25. A method as claimed in claim 1 wherein the amount of compound administered is between 0.5 and 30 grams, typically 3 to 5 grams, per day.

26. A method of treating a patient in need of therapy for dysregulation of TNF-α comprising administering to that patient a therapeutically effective dose of a monoglyceride of general formula I 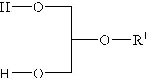
wherein R¹ is the fatty acyl group of an essential polyunsaturated fatty acid selected from γ-linolenoyl, γ-dihomolinolenoyl.

27. A composition for use in the method of the claim 26 comprising a compound of formula I together with a pharmaceutically or nutraceutically acceptable carrier, coating, capsule, diluent and/or preservative.

28. Use of a compound of formula I as described in claim 26 for the manufacture of a medicament for the treatment of dysregulation of cytokines TGF-α or for the modulation of this cytokine.
